(19) United States
(12) Patent Application Publication
(10) Pub. No.: US 2015/0305691 A1
(43) Pub. Date: Oct. 29, 2015

(54) METHOD AND MAGNETIC RESONANCE APPARATUS FOR IMAGE MONITORING OF A MEDICAL INTERVENTIONAL PROCEDURE

(71) Applicant: Siemens Aktiengesellschaft, Muenchen (DE)

(72) Inventor: Eva Rothgang, Nuernberg (DE)

(73) Assignee: Siemens Aktiengesellschaft, Muenchen (DE)

(21) Appl. No.: 14/695,507

(22) Filed: Apr. 24, 2015

(30) Foreign Application Priority Data
Apr. 24, 2014 (DE) 102014207699.4

(51) Int. Cl. A61B 5/00; A61B 19/00; A61B 5/055

(52) U.S. Cl. CPC A61B 5/748; A61B 5/055; A61B 5/7425; A61B 19/5225; A61B 2019/5236

(57) ABSTRACT

In a method and magnetic resonance apparatus for image monitoring of an invasive interventional procedure on a patient with the magnetic resonance apparatus, during the interventional procedure at least two current magnetic resonance monitoring images of the interventional procedure area are repeatedly acquired with different image recording parameters, and at least portions thereof are displayed on a monitor arranged on or as part of the magnetic resonance apparatus. A touchscreen is used as the monitor, on which a segment of an overall display that includes all current monitoring images is displayed. With an operating action involving touching the touchscreen with at least one finger and moving the finger in a direction of movement, the segment is oppositely displaced in a permitted direction of displacement closest to the direction of movement.

### BACKGROUND OF THE INVENTION

1. Field of the Invention

The invention concerns a method for image monitoring of an invasive medical interventional procedure on a patient, such as a minimally invasive procedure, using a magnetic resonance apparatus, wherein during the interventional procedure at least two current magnetic resonance monitoring images of the interventional procedure area are repeatedly acquired with different image recording parameters and at least in part are displayed on a monitor arranged on or as part of the magnetic resonance apparatus. The invention additionally relates to a magnetic resonance apparatus and a non-transitory storage medium encoded with programming instructions to implement such a method.

2. Description of the Prior Art

Magnetic resonance apparatuses are widely known and are in widespread use in the field of medical imaging. Accordingly it has also been proposed, as part of image monitoring using magnetic resonance imaging, to perform medical interventional procedures on a patient, in particular minimally invasive interventional procedures using a catheter and/or a needle. For example, percutaneous interventional procedure needles can be positioned using real-time multi-slice magnetic resonance monitoring. To satisfy the purpose of the image monitoring, the magnetic resonance monitoring images that are acquired, referred to below as monitoring images for short, must be displayed within the mostly shielded area in which the magnetic resonance apparatus is also situated.

In this case the monitoring images must be displayed on a monitor that is large enough to monitor sensitive structures, the target area, and if necessary the trajectory of the instrument used. Furthermore, in many cases the person performing the interventional procedure must monitor the placement of the instrument and/or the progress of the treatment in real-time images that have a different orientation.

Many monitors that can be used within the area containing the magnetic resonance apparatus are limited in size for cost reasons. The cost of the magnetic shielding increase significantly with the size of the display. For example, a magnetic resonance apparatus has been proposed in which a small monitor is arranged in the covering of the main magnet unit above the patient aperture.

Because of the small size of the monitors, it is usually expedient to display only one of the monitoring images within the area in which the magnetic resonance apparatus is arranged. The switch between the individual monitoring images is currently performed by a technician in the monitoring area of the magnetic resonance system. The person performing the interventional procedure gives commands, usually by voice, which are used by the technician to adjust the correct monitoring image which is to be displayed within the area containing the magnetic resonance apparatus. Data acquisition parameters can also be adjusted in this case.

To permit monitoring in real time, the monitoring images are usually recorded using a fast magnetic resonance sequence, in particular with lower resolution and/or lower contrast than would be used for the type of diagnostic image that is to be studied with scrutiny. Thus for example fast fluoroscopy sequences are known for magnetic resonance imaging.

Because of the small monitors available, particularly the small monitors integrated into the magnetic resonance apparatus itself, and the wishes of the person performing the interventional procedure regarding the monitoring image to be displayed, which frequently change during the interventional procedure, an enormous amount of communication has to take place, which is laborious and prone to error and can increase the interventional procedure time.

### SUMMARY OF THE INVENTION

An object of the invention is to provide for easily switching between different monitoring images during an invasive interventional procedure in a magnetic resonance apparatus, particularly a minimally invasive interventional procedure.

This object is in accordance with the invention by a method of the type described above wherein a touchscreen is used as a monitor, on which a segment of an overall display at which all current monitoring images are displayed, and wherein, in response to an operating action in which the touchscreen is touched by at least one finger and the finger is moved in a direction of movement, the segment is oppositely displaced in a permitted direction of displacement closest to the direction of movement.

The invention makes use of a touchscreen, of the type generally known in the prior art. Touchscreens have a touch-sensitive surface that can detect operating gestures performed on the monitor itself. In accordance with the present invention, involves an extremely simple operating concept using such a touchscreen, in which the user navigates in an overall display in which ultimately he or she moves the operating display using simple operating actions, such that the segment actually to be seen on the monitor shows the monitoring image that the user currently needs. To do this, the user merely needs to place a finger on the touchscreen and make a movement in the direction in which the envisaged displacement of the overall display is to take place, which is detected by the detection facility of the touchscreen. The scroll directions (displacement directions) can of course be restricted, as described below. The displacement of the displayed segment is in this case opposite to that of the envisaged movement of the overall display with the finger, so that the displacement direction (scroll direction), which relates to the segment, is also opposite to the direction of movement. Consequently when locating the closest permitted displacement direction for the segment, the direction opposite to the direction of movement should be taken into account.

It is of course the case that the segment is not displaced over the edge of the overall display, such prevention also being known generally in the prior art. If, for example, the segment is already located at the right edge of the overall display and an operating action takes place with the direction of movement leftward, the segment cannot be displaced any further to the right and the displacement is suppressed (not enacted).

It is particularly expedient for the segment to be displaced by the extent of exactly one monitoring image. Then it is only complete monitoring images, preferably exactly one monitoring image, that are ever displayed on the monitor at least in the normal operating mode described here. As a result of the operating action, a user can easily and intuitively switch between the individual monitoring images, without the need for extensive communication with a technician in the monitoring room of the magnetic resonance system.

Overall, therefore, a novel user interaction for magnetic-resonance-managed interventional procedures is described, which allows the user to make use of one finger as an input actuator. This is incidentally also readily possible in a sterile environment, for example if the touchscreen or monitor is covered by a sterilizeable foil or other coating. The interaction display concept described herein permits the use of a relatively small monitor, which keeps shielding costs lower. Nevertheless, the invention makes it possible to switch quickly and easily between different monitoring images that are displayed. No further communication is necessary between the person performing the interventional procedure and a technician outside the room containing the magnetic resonance apparatus, in order to switch between monitoring images, such as slice images with different orientations and planes. The method is highly intuitive and it is not necessary to press any buttons.

In an embodiment of the invention, the overall display can be an arrangement of the monitoring images in a matrix, with directions along the rows and/or columns of the matrix and/or diagonal directions being used as displacement directions for the switching described above. The monitoring images are consequently expediently implemented within the overall display as a type of two-dimensional grid, arranged in rows and columns of a matrix. Using corresponding operating actions to the right and left, or also up and down in the case of a matrix having several rows, the corresponding adjacent monitoring image can be retrieved by means of a single, intuitive operating gesture. Diagonal displacements can of course also be permitted.

In this context it is also particularly expedient for, in the segment displayed, a schematic overview of the overall display to be overlaid, the overview being organized in the form of a matrix. For example, the various monitoring images can be marked in their arrangement by rectangles, with the at least one currently displayed monitoring image, i.e. the segment, being represented by highlighting, for example white highlighting, within the overview. This further simplifies navigation.

Particularly in connection with the arrangement of the monitoring images in a matrix, but also generally, it may be expedient to save the monitoring images in a single file in DICOM format. Such a file can be called a “mosaic file”, and contains the matrix of the individual monitoring images which themselves may represent DICOM images. The file, or generally the overall display, is updated after every recording of new monitoring images. For example, a new recording of monitoring images can take place whenever the instrument is moved, and/or cyclically and/or after an EKG and/or respiratory triggering, so that consequently also the overall display, in particular the file, is also updated and consequently can be captured as a real-time overall display.

As noted, in a preferred embodiment of the invention the segment always contains exactly one monitoring image. In this way the existing, restricted display space is utilized as fully as possible on the monitor. Exemplary embodiments are also conceivable, in which at least two complete monitoring images are displayed, thought this is less preferred in the case of small monitors.

At least some of the monitoring images can show an instrument used during the interventional procedure and/or the target area. For example, in interventional procedures with a needle, it is known for two slice images to be selected, on which the penetration depth of the needle can readily be identified. Consequently it is generally expedient, when a needle is used as an instrument, for at least some of the monitoring images to show a penetration depth of the instrument.

At least one further monitoring image can relate to the target area, so that for example it is possible to quickly identify when the instrument, in particular the needle, actually reaches the target area.

As already noted, at least some of the monitoring images can be two-dimensional slice images, in particular slice images which are at least in part in different orientations. Thus for example the target area and/or the instrument can be monitored from different orientations.

Since in the inventive method a touchscreen is used in any case, it is expedient to permit further operating actions which provide further functions. For example, after a zoom gesture is detected, a magnification level of the segment can be adjusted. Such a zoom gesture may be, for example, tapping the touchscreen twice, but it is also possible for the touchscreen to be designed so as to be able to detect the touches of several fingers independently (i.e. it is multitouch-capable), and to interpret two fingers being moved apart and/or together on the touchscreen as a zoom gesture. Such zoom gestures already known in principle in the prior art. Particularly if the segment normally includes exactly one monitoring image, it is possible to switch from a normal operating mode when a zoom gesture is detected to a zoom operating mode, in which navigation to the monitoring image currently selected in the segment is reduced. This is because boosting the magnification level means that a sub-segment of the segment, in other words of a particular monitoring image, is displayed, it being possible to move the sub-segment within the segment ultimately in exactly the same way as the segment itself within the overall display, so that the operating concept in respect of the overall display, preferably as a continuous displacement facility, can be transferred to a monitoring image in the zoom operating mode.

In this case, the overall display need not include only continuously updated monitoring images in all exemplary embodiments of the inventive method, but provision can also be made for at least one planning image recorded and/or generated before the interventional procedure to be integrated into the overall display. It is frequently the case that pre-interventional procedure image recordings are made, with the interventional procedure being planned in the correspondingly contained planning images. It may now be expedient, even during the interventional procedure itself, to refer back to these planning images, which can accordingly be recorded in the overall display, for example can be integrated into a corresponding matrix.

Besides the method, the invention also relates to a magnetic resonance apparatus, having a monitor designed as a touchscreen that is integrated into the magnetic resonance apparatus, in particular arranged at the end face of the magnetic resonance apparatus above the opening of the patient aperture, and a control computer designed to perform the inventive method. All embodiments relating to the inventive method apply analogously to the inventive magnetic resonance apparatus, with which the advantages already described can consequently also be obtained. The control computer controls the acquisition of the monitoring images and correspondingly updates the overall display. Via the touch-sensitive surface of the touchscreen, it detects the operating action and adjusts the segment accordingly. As described, other functions can also be implemented.

The present invention also encompasses a non-transitory, computer-readable data storage medium encoded with programming instructions that, when the storage medium is loaded into a control computer of a magnetic resonance apparatus, cause the magnetic resonance apparatus to be operated so as to implement the method as described above in accordance with the invention.

The non-transitory data storage medium can be, for example, a CD-ROM.

### BRIEF DESCRIPTION OF THE DRAWINGS

FIG. 1 shows an inventive magnetic resonance apparatus.

FIG. 2 shows an overall display with a segment to be displayed in a first exemplary embodiment of the invention.

FIG. 3 shows the overall display from FIG. 2 with a displaced segment of the invention.

FIG. 4 shows an overall display with a segment to be displayed in a second exemplary embodiment of the invention.

FIG. 5 shows an example of a display on a monitor of the invention.

### DESCRIPTION OF THE PREFERRED EMBODIMENTS

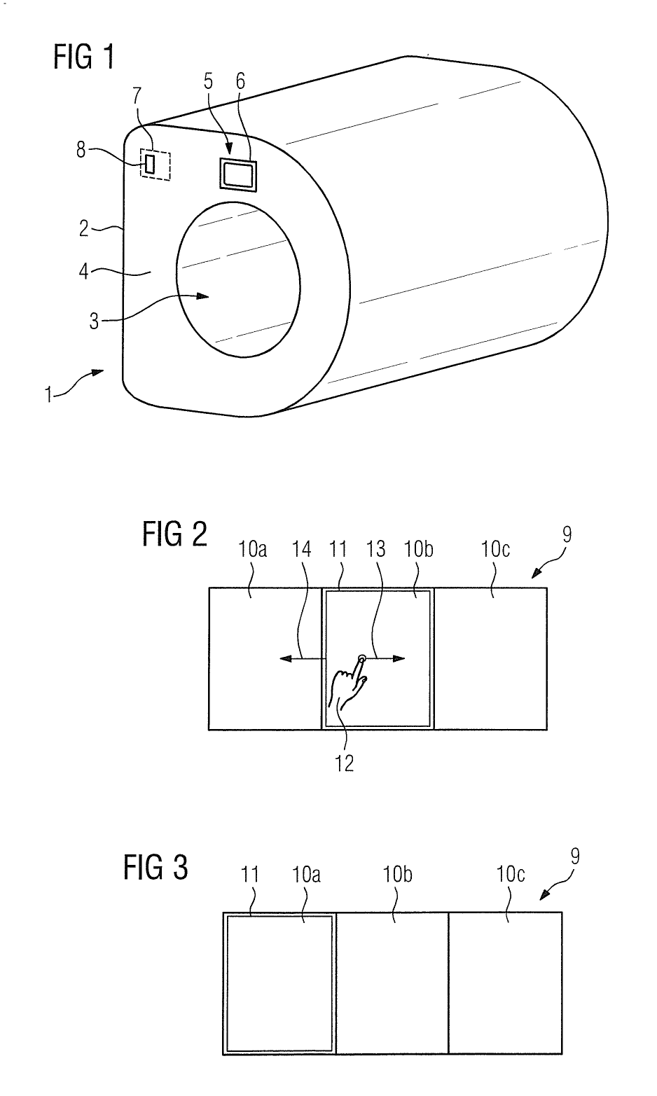

FIG. 1 is a block diagram of an inventive magnetic resonance apparatus 1. As is known in principle, the magnetic resonance apparatus 1 has a scanner 2 that defines a patient aperture 3, into which a patient can be introduced for magnetic resonance imaging by a patient bed (not shown in detail). A small monitor 5 is integrated into the covering or exterior shell 4 of the scanner 2 above the patient aperture, and in this case is implemented as a touchscreen 6, and consequently has a touch-sensitive surface. The monitor 5 is designed to be very small and can be used during a minimally invasive interventional procedure using image monitoring by the magnetic resonance apparatus 1, in order to display recorded monitoring images. A single monitoring image, for example a fast fluoroscopy scan in a particular orientation, is always displayed as a segment of an overall display of all respective monitoring images to be currently recorded, it being possible to navigate within the overall display using simple operating actions, in which at least one finger is placed on the touchscreen 6 and is moved in a direction of movement in order to move the segment in the overall display in a direction of displacement opposite to this direction of movement, if possible, by exactly one monitoring image, so that the monitoring image adjacent in the opposite direction is displayed. Consequently the impression is given that the user is using his or her finger to drag the overall display lying behind the monitor 5.

The operation of the magnetic resonance apparatus 1 and consequently also of the monitor 5 is controlled by a control computer 7 of the magnetic resonance apparatus 1, which is designed to perform the inventive method. This means that the control computer 7 triggers the other components of the magnetic resonance apparatus 1 to acquire current monitoring images and can determine the respective current overall display. Furthermore, operating actions/gestures can be detected on the touchscreen 6 and evaluated, in order to update the display on the monitor 5, by displaying a correspondingly different/amended segment.

In this case the overall display is stored as a separate, single DICOM file 8 in a memory of the control computer 7. Thus the most up-to-date monitoring images are always available within a single file 8, it being possible to navigate using the touchscreen 6 within the overall display stored in the file 8.

The monitoring images which were recorded with different recording parameters represent different aspects of the interventional procedure area, and are thus, in particular, slice images with different orientations. If need be, and as can be selected by a user, pre-interventional procedure images, in particular planning images, can be integrated into the overall display, in which case it is expedient to also have them available during the minimally invasive interventional procedure.

In the present case a zoom function can also be implemented via the control computer 7, so that when a zoom gesture is detected, for example tapping the touchscreen 6 twice, the system switches to a zoom operating mode, in which it is possible to navigate as smoothly as possible within the monitoring image precisely contained in the segment; specifically a sub-segment of the segment is thus displayed, it being possible to move the sub-segment using the same operating actions, but without restricted directions of displacement (scroll directions).

FIGS. 2 and 3 explain the operating concept again in greater detail using a first exemplary embodiment, in which a schematically displayed overall display 9 contains three monitoring images 10a, 10b and 10c. The segment 11, represented by a box with a thickened outline or border, can be seen over the central monitoring image 10b. In the case of a minimally invasive interventional procedure with a needle, the monitoring images 10a and 10b can be, for example, slice images, in which the penetration depth of the needle is shown, whereas the monitoring image 10c is a slice image of the target region. The monitoring images 10a, 10b, 10c are apparent, arranged in a row in a here one-dimensional matrix, so that the permissible directions of displacement inside the overall display 9 are to the right or left. Consequently if a user touches the touchscreen 6 with his or her hand 12, for example with the index finger, and then moves the index finger in the direction of movement 13, here to the right, this operating action is detected by the control computer 7 and the segment 11 is moved in the opposite scroll direction 14, i.e. to the left, by an amount equal to one monitoring image 10a, 10b, 10c. The status in FIG. 3 is then obtained, in which the segment can be seen over the monitoring image 10a.

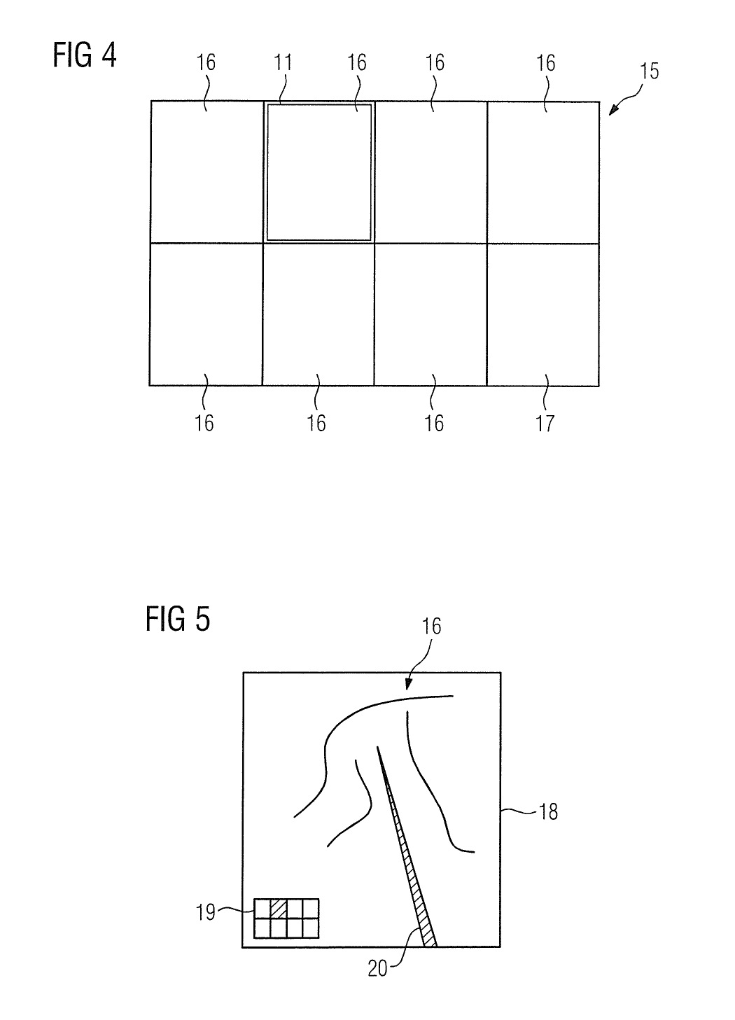

FIG. 4 shows a further overall display 15 of a second exemplary embodiment, which in this case has seven monitoring images 16 arranged in a two-dimensional matrix and a planning image 17. The individual images are here arranged in two rows and four columns. Other expedient directions of displacement are “up” and “down”, and are correspondingly implemented. The implementation of diagonal directions as permitted directions of displacement is also conceivable.

FIG. 5 shows an example of a display 18 on the monitor 5, which includes not only the image data for the monitoring image 16 in the segment 11, but also a schematic overview 19 that shows the current position in the matrix of the overall display 15 in greater detail. The needle 20 is incidentally clearly identifiable in the monitoring image 16 of the display 18 as an instrument in relation to the anatomy.

Although modifications and changes may be suggested by those skilled in the art, it is the intention of the inventor to embody within the patent warranted hereon all changes and modifications as reasonably and properly come within the scope of her contribution to the art.

I claim as my invention:

1. A method for image monitoring of a medical intervention on a patient situated in a magnetic resonance (MR) scanner, comprising:
during said medical intervention, operating said MR scanner to acquire at least two current MR monitoring images of a region of the patient in which said intervention proceeds, by operating said MR scanner with respectively different image acquisition parameters respectively for said at least two current MR monitoring images;
displaying said at least two current MR images on a monitor having a touchscreen on, or forming a part of, an exterior housing of said MR scanner;
on said touchscreen, also displaying a segment that encompasses only a portion of an overall screen area of said touchscreen; and
operating said monitor to respond to an operating action comprising touching said touchscreen with at least one finger within said segment and moving said finger in a direction of movement and thereby causing said segment to be oppositely displaced on said touchscreen in a permitted direction of displacement that is closest to said direction of movement of the finger.

2. A method as claimed in claim 1 comprising displaying said at least two current MR monitoring images at said touchscreen each with an image with, and displacing said segment, in response to said movement of the finger, by an amount equal to said image width.

3. A method as claimed in claim 1 comprising displaying said at least two current MR monitoring images at said touchscreen in a matrix comprised of rows and columns, and using at least one of a row direction or a column direction or a diagonal direction as said permitted direction of displacement.

4. A method as claimed in claim 3 comprising operating said monitor from a computer having access to an electronic memory and storing said at least two current images in said electronic memory in a single file in DICOM format.

5. A method as claimed in claim 4 comprising, via said computer, updating said file upon each acquisition of a new current MR monitoring image.

6. A method as claimed in claim 1 comprising configuring said segment to always encompass exactly one of said current MR monitoring image at said touchscreen.

7. A method as claimed in claim 1 comprising configuring said segment to always encompass at least two of said current MR monitoring images at said touchscreen.

8. A method as claimed in claim 1 comprising operating said MR scanner to acquire at least one of said current MR monitoring images to show an instrument used in said intervention.

9. A method as claimed in claim 1 comprising operating said MR scanner to acquire at least one of said MR monitoring images to show a target area of said intervention.

10. A method as claimed in claim 9 comprising employing a needle to conduct said intervention, and comprising acquiring at least one of said current MR monitoring images to show a penetration depth of said needle in the subject with respect to a target area of said intervention.

11. A method as claimed in claim 1 comprising operating said MR scanner to acquire at least some of said current MR monitoring images as slice images of the subject.

12. A method as claimed in claim 11 comprising operating said MR scanner to acquire said slice images with respectively different orientations.

13. A method as claimed in claim 1 comprising configuring said touchscreen to respond to a zoom gesture by enlarging said segment.

14. A method as claimed in claim 1 comprising also displaying at said touchscreen a planning image of the subject, acquired prior to said at least two current MR monitoring images.

15. A magnetic resonance (MR) apparatus comprising:
an MR scanner comprising an exterior scanner housing;
a display monitor comprising a touchscreen at, or formed as part of, said exterior scanner housing;
a control computer configured to operate said MR scanner during said medical intervention to acquire at least two current MR monitoring images of a region of the patient in which said intervention proceeds, by operating said MR scanner with respectively different image acquisition parameters respectively for said at least two current MR monitoring images;
said control computer being in communication with said display monitor and being configured to display said at least two current MR images on said touchscreen on;
said control computer being configured to also display on said touchscreen a segment that encompasses only a portion of an overall screen area of said touchscreen; and
said control computer being configured to operate said monitor to respond to an operating action comprising touching said touchscreen with at least one finger within said segment and moving said finger in a direction of movement and thereby causing said segment to be oppositely displaced on said touchscreen in a permitted direction of displacement that is closest to said direction of movement of the finger.

16. A non-transitory, computer-readable data storage medium encoded with programming instructions, said storage medium being loaded into a control computer of a magnetic resonance (MR) apparatus, said MR apparatus comprising an MR scanner having an exterior housing and a display monitor comprising a touchscreen at, or formed as part of, said exterior scanner housing, said programming instructions causing said control computer to:
during a medical intervention, operate said MR scanner to acquire at least two current MR monitoring images of a region of the patient in which said intervention proceeds, by operating said MR scanner with respectively different image acquisition parameters respectively for said at least two current MR monitoring images;
display said at least two current MR images on said touchscreen;
on said touchscreen, also display a segment that encompasses only a portion of an overall screen area of said touchscreen; and
operate said monitor to respond to an operating action comprising touching said touchscreen with at least one finger within said segment and moving said finger in a direction of movement and thereby causing said segment to be oppositely displaced on said touchscreen in a permitted direction of displacement that is closest to said direction of movement of the finger.
